(19) United States
(12) Patent Application Publication
(10) Pub. No.: US 2023/0066875 A1
(43) Pub. Date: Mar. 2, 2023

(54) TRANSDUCER APPARATUSES WITH ELECTRODE ARRAY SHAPED TO REDUCE EDGE EFFECT IN DELIVERING TUMOR TREATING FIELDS TO A SUBJECT'S BODY

(71) Applicant: Novocure GmbH, Root D4 (CH)

(72) Inventors: Tal MARCIANO, Haifa (IL); Smadar ARVATZ, Haifa (IL); Boaz MARSAULT, Haifa (IL)

(73) Assignee: Novocure GmbH, Root D4 (CH)

(21) Appl. No.: 17/886,382

(22) Filed: Aug. 11, 2022

Related U.S. Application Data
(63) Continuation of application No. 17/698,457, filed on Mar. 18, 2022.
(60) Provisional application No. 63/232,361, filed on Aug. 12, 2021.
(60) Provisional application No. 63/232,329, filed on Aug. 12, 2021.

(51) Int. Cl. A61N 1/40

(52) U.S. Cl. CPC A61N 1/40

(57) ABSTRACT

A transducer apparatus for delivering tumor treating fields to a subject's body, the transducer apparatus including: an array of electrode elements electrically coupled to each other, the array including all electrode elements present on the transducer apparatus, the array configured to be positioned over the subject's body with a face facing the subject's body; wherein, when viewed from a direction perpendicular to the face of the array, an outer perimeter of the array substantially tracing the electrode elements of the array has a rounded convex shape; a number of the electrode elements of the array are peripheral electrode elements defining the outer perimeter of the array, the peripheral electrode elements substantially surrounding any other electrode elements of the array; and wherein for each peripheral electrode element, at least a portion of a length of a perimeter of the peripheral electrode element is touching the outer perimeter of the array.

### CROSS REFERENCE TO RELATED APPLICATIONS

This application claims priority to U.S. patent application Ser. No. 17/698,457 filed Mar. 18, 2022, U.S. Patent Application No. 63/232,329 filed Aug. 12, 2021, and U.S. Patent Application No. 63/232,361 filed Aug. 12, 2021, all of which are incorporated herein by reference.

### BACKGROUND

Tumor treating fields (TTFields) are low intensity (e.g., 1-4 V/cm) alternating electric fields within the intermediate frequency range (e.g., 50 kHz to 1 MHz, such as 50-550 kHz), which may be used to treat tumors as described in U.S. Pat. No. 7,565,205. TTFields therapy is an approved mono-treatment for recurrent glioblastoma (GBM) and an approved combination therapy with chemotherapy for newly diagnosed GBM patients. TTFields can also be used to treat tumors in other parts of a subject's body (e.g., lungs, ovaries, pancreas). For example, TTFields therapy is an approved combination therapy with chemotherapy for malignant pleural mesothelioma (MPM). TTFields are induced non-invasively into the region of interest by transducers (e.g., arrays of capacitively coupled electrode elements) placed directly on the patient's body (e.g., using the Novocure Optune™ system), and applying AC voltages between the transducers.

Conventional transducers used to generate TTFields include a plurality of ceramic disks. One side of each ceramic disk is positioned against the patient's skin, and the other side of each disc has a conductive backing. Electrical signals are applied to this conductive backing, and these signals are capacitively coupled into the patient's body through the ceramic discs. Conventional transducer designs include rectangular arrays of ceramic disks aligned with each other in straight rows and columns (e.g., in a three-by-three arrangement).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts an example of transducers located on a subject's head.

FIG. 2 depicts an example of transducers located on a subject's torso.

FIGS. 3A and 3B depict cross-sectional views of examples of the structure of various transducers.

FIG. 3C depicts a thermal image of a conventional rectangular electrode array of ceramic disks aligned with each other in straight rows and columns in a three-by-three arrangement.

FIG. 4 depicts an example layout of an array of electrode elements on a transducer apparatus.

FIG. 5 depicts an electrode element of the array of FIG. 4.

FIG. 6 depicts another example layout of an array of electrode elements on a transducer apparatus.

FIG. 7 depicts another example layout of an array of electrode elements on a transducer apparatus.

FIG. 8 depicts another example layout of an array of electrode elements on a transducer apparatus.

FIG. 9 depicts another example layout of an array of electrode elements on a transducer apparatus.

FIG. 10 depicts another example layout of an array of electrode elements on a transducer apparatus.

FIGS. 11A-11C depict examples of the electric field strength from arrays of electrode elements having different shapes.

FIG. 12 depicts a plot of average power loss with respect to array surface area for electrode arrays having different outer perimeter shapes.

Various embodiments are described in detail below with reference to the accompanying drawings, wherein like reference numerals represent like elements.

### DESCRIPTION OF EMBODIMENTS

This application describes exemplary transducer apparatuses for delivering TTFields to a subject's body and used to treat one or more cancers (tumors) located in the subject's body.

When TTFields are applied to a subject's body, the temperature at the subject's body may increase proportionally to the induced electric field. Regulations limit the amount of current that can be driven through a transducer to an amount that keeps the measured temperature at locations on the subject's body below a temperature threshold. As practiced in the art, the temperature at the location of the transducers on the subject's body is controlled to be below the temperature threshold by reducing the operational current driven by the transducer and reducing the strength of the resulting TTFields. This in turn becomes an over-riding limitation on the TTFields strength that can be used to treat the tumor. Accordingly, there is a need in the art to safely access higher TTField strengths without exceeding the temperature threshold at the subject's skin.

The inventors have discovered that, on a transducer comprising an array of electrode elements, the electrode elements located along the edge of the array have a lower resistance to current flowing therethrough compared to the electrode elements located toward the middle of the array. This can lead to higher concentrations of electric charge at points on the edge (e.g., outer perimeter) of the array in general. Further, an electrode element located at a corner or similar sharp bend in the edge of the array will have a higher concentration of electric charge than other electrode elements along the edge and in the center of the array. The tendency of a transducer to drive higher amounts of current through electrode elements located along the edge of the array, and particularly at the corners, is referred to herein as the “edge effect.”

An uneven distribution of current through the array of a transducer due to the edge effect can lead to higher temperature zones (or “hot spots”) forming at distant corners and along edges of the array. These hot spots are the locations that reach the threshold temperature first and therefore control the requirement to reduce the current. As such, the generation of hot spots due to the edge effect limits the maximum operational current that may be driven by a transducer, and the strength of the resulting TTFields.

The inventors have now recognized that a need exists for transducers having electrode element array layouts that reduce or minimize the edge effect and allow the application of higher operating currents to the transducers. Transducers operated with increased current can induce stronger TTFields in the subject's body, ultimately leading to better patient outcomes. Each of the disclosed transducer apparatuses have an array of electrode elements positioned in a layout and having shapes that reduce or minimize the edge effect.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

As used in the specification and the appended claims, the singular forms “a,” “an” and “the” include plural referents unless the context clearly dictates otherwise.

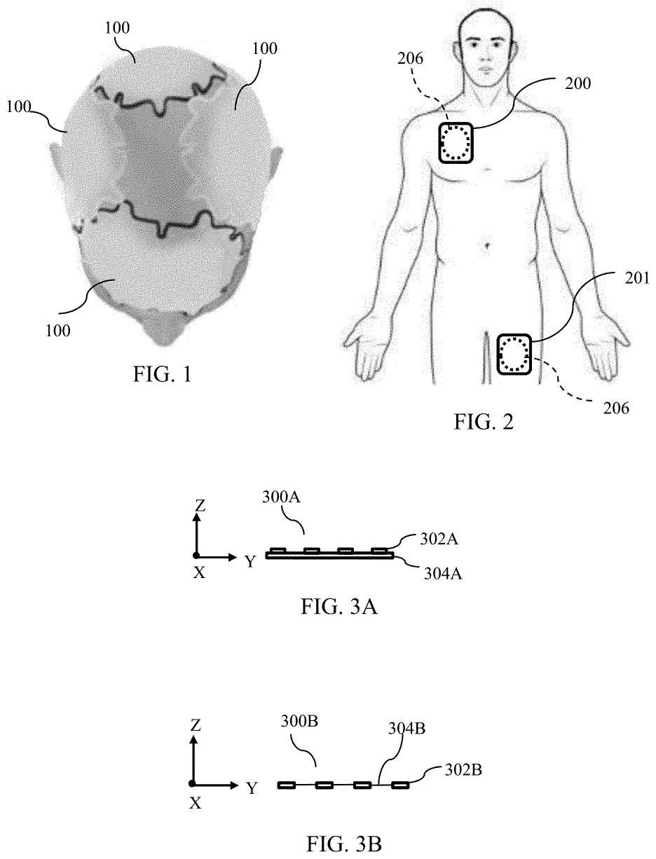

FIG. 1 depicts transducers 100 positioned on the head of a subject's body. FIG. 1 depicts one example of a subject's head on which transducers 100 are placed in various positions and/or orientations. Such an arrangement of transducers 100 on a subject's head is capable of applying TTFields to a tumor in a region of the subject's brain. It should be noted that various other positions and/or orientations on the subject's head may be selected for placement of transducers.

Each transducer 100 may have an array of electrode elements disposed thereon as described herein. Each transducer 100 may be placed on a subject's head with a face of the array of electrode elements facing the subject's head. A transducer 100 may be placed on the subject's head such that the face of the array of electrode elements conforms to the outer shape of the head.

FIG. 2 depicts first and second transducers positioned at first and second locations, respectively, on the torso of a subject's body. Specifically, FIG. 2 depicts a first transducer 200 located on the front of the subject's right thorax and a second transducer 201 located on the front of the subject's left thigh. It should be noted that various other locations on the subject's torso may be selected for placement of one or more pairs of transducers.

FIG. 2 depicts the transducers 200 and 201 attached to the subject's body. As an example, the transducers 200 and 201 may be affixed to the subject's body by applying a medically appropriate adhesive onto a surface of each transducer. In other embodiments, the transducers 200 and 201 may be attached to one or more garments (not shown) such as, for example, a shirt and pants. In an example, the transducers 200 and 201 may be attached to clothes using adhesive. In another example, the transducers 200 and 201 may be attached to clothes by incorporating the transducers 200 and 201 within the clothing. In examples where transducers are disposed at locations on the subject's head, the corresponding transducers may be integrated in another type of garment (e.g., hat).

Each of the transducers 200 and 201 may have an array of electrode elements disposed thereon as described herein. Each transducer 200 and 201 may be placed over the subject's body with a face of the array of electrode elements facing the subject's body. The transducers 200 and 201 may be placed on the subject's body such that the face of the corresponding array of electrode elements conforms to the outer shape of the subject's body.

In both the first transducer 200 and the second transducer 201, the array of electrode elements may be arranged and located within an outer perimeter 206 (defined by a dashed line in FIG. 2). In an example, the outer perimeter 206 of the array on each transducer may have a substantially rounded edge. In an example, the outer perimeter 206 of the array on each transducer may be substantially circular, oval, ovaloid, ovoid, or elliptical in shape. Other shapes of the outer perimeter 206 may be possible as well.

The arrays of electrode elements may include a number of different layouts disclosed herein that reduce or minimize the edge effect during operation of the transducers. The layouts may include, for example, one or more of: peripheral electrode elements shaped to conform to a rounded outer perimeter 206; a certain percentage of the length of a rounded outer perimeter 206 touching the electrode elements of the array; a peripheral electrode element shaped to touch at least a certain percentage of the length of a rounded outer perimeter 206; and/or electrode elements of the array each being disposed along or adjacent the outer perimeter 206.

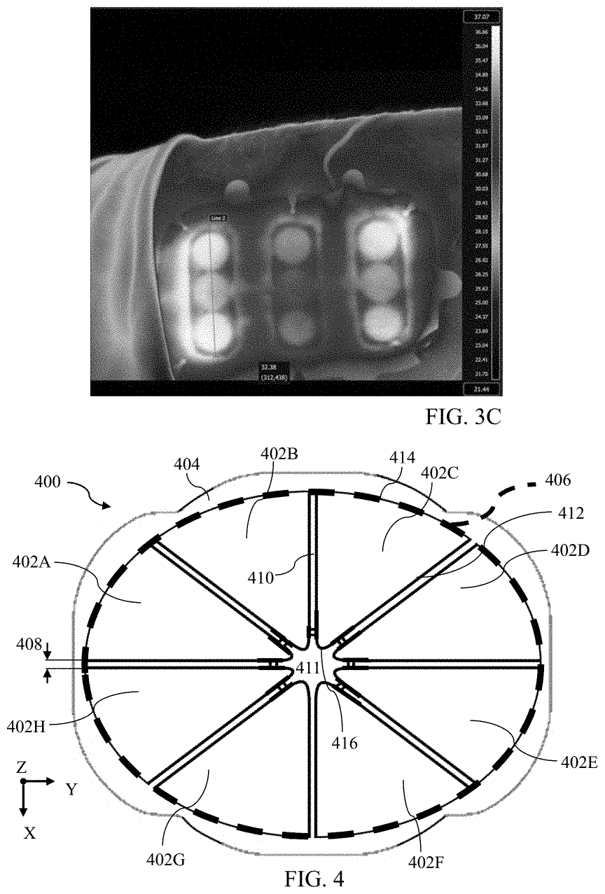

FIGS. 3A and 3B depict cross-sectional views of examples of the structure of a transducer. For example, as shown in FIG. 3A, the transducer 300A has a plurality of electrode elements 302A and a substrate 304A. The substrate 304A is configured for attaching the transducer 300A to a subject's body. Suitable materials for the substrate 304A include, for example, cloth, foam, and flexible plastic. In one example, the substrate 304A includes a conductive medical gel having a thickness of not less than approximately 0.5 mm. In a more specific example, the substrate 304A is a layer of hydrogel with a minimum thickness of 0.5 mm. In this situation, the transducer 300A is attached to the subject's body through the substrate 304A.

A plurality of electrode elements 302A are positioned on the substrate 304A. Each of the electrode elements may have a conductive plate with a dielectric layer disposed thereon that faces towards the substrate 304A. Optionally, one or more sensors may be positioned beneath each of the electrode elements 302A in a manner that is similar to the conventional arrangement used in the Novocure Optune® system. In one example, the one or more sensors are temperature sensors (e.g., thermistors).

FIG. 3B depicts a cross-sectional view of another example of the structure of the transducer 300B. In this example, the transducer 300B includes a plurality of electrode elements 302B. The plurality of electrode elements 302B are electrically and mechanically connected to one another without a substrate. In one example, the electrode elements 302B are connected to one another through conductive wires 304B.

As depicted in FIGS. 3A and 3B, the transducers 300A and 300B comprise arrays of substantially flat electrode elements 302A and 302B, respectively. In each of FIGS. 3A and 3B, the array of electrode elements may be capacitively coupled. In some embodiments, the electrode elements 302A and 302B are non-ceramic dielectric materials positioned over a plurality of flat conductors. Examples of non-ceramic dielectric materials positioned over flat conductors include polymer films disposed over pads on a printed circuit board or over flat pieces of metal. In other embodiments, the electrode elements 302A and 302B are ceramic elements.

Transducers that use an array of electrode elements that are not capacitively coupled may also be used. In this situation, each electrode element 302A and 302B may be implemented using a region of a conductive material that is configured for placement against a subject's body, with no insulating dielectric layer between the conductive elements and the body.

Other alternative constructions for implementing the transducer for use with embodiments of the invention may also be used, as long as they are capable of (a) delivering TTFields to the subject's body and (b) being positioned at locations of the subject's body.

FIGS. 3A and 3B depict the transducers 300A and 300B from a direction perpendicular to a Y-Z plane defined by a 3-dimensional coordinate axis shown in the figures. As illustrated, the electrode elements 302A and 302B are distributed along a direction parallel to the Y-axis. In addition, the electrode elements 302A and 302B may be distributed along a direction parallel to the X-axis. As such, the transducers 300A and 300B may each comprise an array of electrode elements 302A and 302B, respectively, distributed along a face of the array in a plane parallel to the X-Y plane. The face of the array (parallel to the X-Y plane) is configured to face the subject's body when the transducer is positioned over the subject's body. Similar 3-dimensional coordinate axes are depicted in the remaining figures.

FIG. 3C depicts a thermal heat map of a 9-electrode transducer array (3×3 rectangular array of electrodes) in use, which illustrates the presence of higher temperature zones, or “hot-spots”, along the edges, and particularly at the corners of the array. As discussed above, the generation of hot spots due to the edge effect limits the maximum operational current that may be driven by a transducer, and the strength of the resulting TTFields.

FIGS. 4 and 6-10 each depict example layouts of electrode elements on a transducer, in accordance with disclosed embodiments. In each example layout of electrode elements described herein (e.g., in FIGS. 4 and 6-10), the layout is viewed from a direction perpendicular to the face (i.e., perpendicular to the X-Y plane) of the array of electrode elements. The array of electrode elements is configured to be positioned over the subject's body with this face of the array facing the subject's body. In each example layout described herein (e.g., in FIGS. 4 and 6-10), the “array of electrode elements” comprises all electrode elements (e.g., 402A-402H in FIG. 4) present on the transducer apparatus (e.g., 400 in FIG. 4).

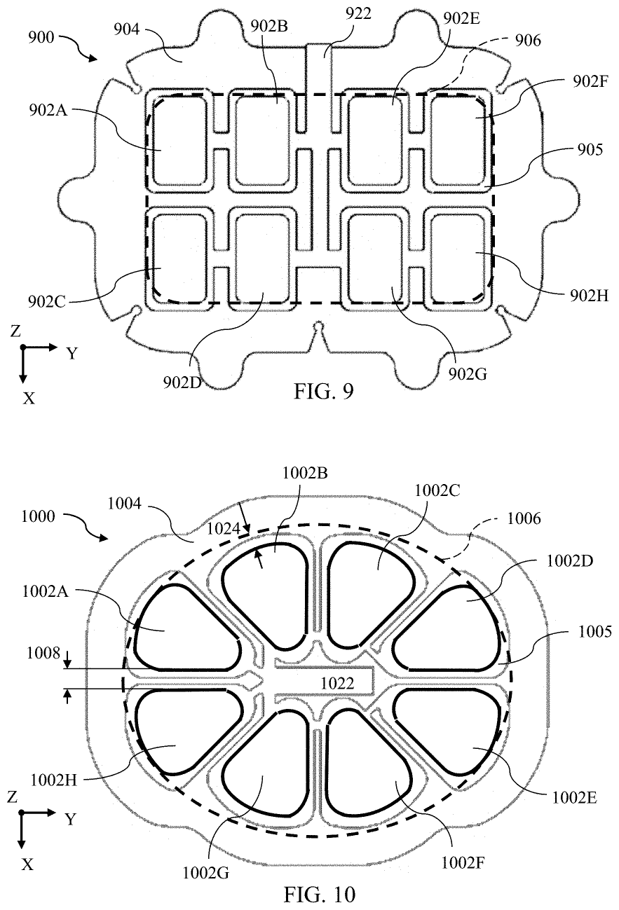

As depicted in FIGS. 4 and 6-10, the transducer (e.g., 400 in FIG. 4) may include a substrate (e.g., 404 in FIG. 4) on which the electrode elements are disposed. In some embodiments (e.g., FIG. 9), the substrate may have cuts, slits, or perforations formed therein to facilitate placement of the substrate over rounded edges of a subject's body. As discussed above, other embodiments of the transducer may not include a substrate. The disclosed electrode element layouts may be equally applied to transducers in which a substrate is present to transducers where no substrate is present.

In each electrode element layout described herein (e.g., in FIGS. 4 and 6-10), a number of the electrode elements of the array are “peripheral electrode elements.” In FIGS. 4, 6, 7, 9, and 10, for example, all the electrode elements present on the transducer are peripheral electrode elements. In another example as shown in FIG. 8, only a subset of the electrode elements (e.g., 802A-802H) on the transducer are peripheral electrode elements. In such embodiments, the peripheral electrode elements (e.g., 802A-802H in FIG. 8) may substantially surround all other electrode elements (e.g., 8021 in FIG. 8) of the array. The term “substantially surround” may refer to a convex shape that passes through the centroids of all peripheral electrode elements surrounding or enclosing every other (non-peripheral) electrode element. In each figure described below, the peripheral electrode elements may define an outer perimeter (e.g., 406 in FIG. 4) of the array of electrode elements. In each figure described below, the array of electrode elements on a transducer includes at least six electrode elements. In an example, the array of electrode elements on a transducer includes at least eight electrode elements.

In several electrode element layouts described herein (e.g., in FIGS. 4 and 6-10), an outer perimeter (e.g., 406 in FIG. 4) of the array substantially tracing the electrode elements of the array has a rounded convex shape. The term “rounded convex shape” refers to any two-dimensional shape that 1) has at least one portion thereof with a radius of curvature (i.e., the shape is at least partially rounded); and 2) does not have any concave portions. In certain transducers, for example as depicted in FIGS. 4, 6-8, and 10, the rounded convex outer perimeter does not have any corners (e.g., sharp corners where two straight edges meet at a point, rounded corners, etc.). The rounded convex outer perimeter may be substantially circular, oval, ovaloid, ovoid, or elliptical, as shown in FIGS. 4, 6-8, and 10. In other transducers, for example as depicted in FIG. 9, the rounded convex outer perimeter 906 has rounded corners. For example, the rounded convex outer perimeter may be substantially rectangular with rounded corners, substantially polygonal with rounded corners, or other convex shapes with rounded corners.

Each electrode element layout described herein (e.g., in FIGS. 4 and 6-10) is designed to reduce or minimize the edge effect and reduce the presence or intensity of hot spots formed at the outer perimeter of the array of electrode elements. This may be accomplished by manipulating the geometry of the overall array (defined by the outer perimeter) of electrode elements, manipulating the geometry of individual electrode elements, and/or making all electrode elements of the array peripheral electrode elements. Setting the geometry of the array of electrode elements in this manner may balance the current output from individual electrodes of the array such that the current is relatively consistent across the array or across the edge of the array. This allows for increasing the current supplied to the transducer while maintaining temperatures on the subject's body below a threshold temperature.

FIG. 4 depicts a transducer 400 with an example layout of electrode elements 402, which may be disposed on a substrate 404. As illustrated, the electrode elements 402 of the transducer 400 are coupled to each other. In FIG. 4, the transducer's array of electrode elements comprises eight electrode elements 402A-402H, all of which are peripheral electrode elements.

FIG. 4 depicts an outer perimeter 406 in dashed lines. The outer perimeter 406 is a rounded convex perimeter substantially tracing the electrode elements 402 of the array, as described above. The outer perimeter 406 may be defined by a form-fit convex shape surrounding the electrode elements 402. Further, as depicted, the outer perimeter 406 circumscribes the array of electrode elements 402. In the embodiment of FIG. 4, the outer perimeter 406 touches an edge of every electrode element 402A-402H in the array.

As depicted in FIG. 4, at least a portion of a length of a perimeter of each peripheral electrode element (e.g., each electrode element 402A-402H) is touching the outer perimeter 406. Each electrode element 402A-402H in FIG. 4 may touch the outer perimeter 406 at more than a single point along the outer perimeter 406. In an example, the outer perimeter 406 traces one or more curved edges (e.g., 414) of the electrode elements 402 touching the outer perimeter 406. In embodiments where all electrode elements 402 and/or all peripheral electrode elements have a length of their perimeter touching/tracing a rounded, convex outer perimeter 406, the current output through the electrode elements 402 may be more effectively balanced.

In an example, the electrode elements 402A-402H in the array may be spaced substantially equidistant from each other about the array. In FIG. 4, for example, each pair of adjacent peripheral electrode elements (e.g., 402A and 402H) touching the outer perimeter 406 has approximately a same distance (e.g., 408) therebetween. More specifically, the distance between a pair of adjacent peripheral electrode elements is not more than 5% greater than a distance between any other pair of adjacent peripheral electrode elements of the array. In other examples, the “substantially equidistant” spacing may refer to a distance between a pair of adjacent peripheral electrode elements being not more than 2% greater, more particularly not more than 1% greater, than a distance between any other pair of adjacent peripheral electrode elements of the array. For example, the distance 408 between electrode elements 402A and 402H in FIG. 4 is not more than 5% greater, particularly not more than 2% greater, and particularly not more than 1% greater than any one of the distances between: electrode elements 402A and 402B; electrode elements 402B and 402C; electrode elements 402C and 402D; electrode elements 402D and 402E; electrode elements 402E and 402F; electrode elements 402F and 402G; and electrode elements 402G and 402H. In an example, the distance between a pair of adjacent electrode elements may be a shortest distance from a point where a first electrode element intersects or touches the outer perimeter 406 to a point where a second adjacent electrode element intersects or touches the outer perimeter 406. In an example, the distance between a pair of adjacent electrode elements may be measured along the length (straight line or arc) of the outer perimeter 406. Arranging the electrode elements 402 to be spaced substantially equidistant from each other along the outer perimeter 406 may balance the electromagnetic shielding between electrode elements 402 of the array, contributing to a more balanced current output.

Certain shapes of the individual electrode elements 402 may also help balance the current through the array. In an example, at least one of the electrode elements 402 in the array may have a triangular shape, a substantially triangular shape with rounded corners, a truncated triangular shape, a substantially truncated triangular shape with rounded corners, a wedge shape, a substantially wedge shape with rounded corners, a truncated wedge shape, or a substantially truncated wedge shape with rounded corners. FIG. 4 depicts each of the electrode elements 402 having a substantially wedge shape with a radially internal facing rounded corner and a radially external facing rounded edge between the two remaining corners. As illustrated with reference to the electrode element 402C, one or more electrode elements 402 may comprise: a first edge 410 extending in a radially outward direction relative to a center portion 411 of the array; a second edge 412 extending in a radially outward direction relative to the center portion 411 of the array; and a rounded edge 414 connecting the first edge 410 to the second edge 412 at an end of the electrode element located radially away from the center portion 411 of the array. As illustrated, a rounded corner 416 may connect the first edge 410 to the second edge 412 at an opposite end of the electrode element located radially toward the center portion 411. Although not shown, in other embodiments the corner connecting the first edge 410 to the rounded edge 414 and the corner connecting the second edge 412 to the rounded edge 414 may each be rounded corners similar to the rounded corner 416. The radius of curvature of the rounded edge 414 may be larger than the radius of curvature of the rounded corner 416.

The shape of the electrode elements 402 in FIG. 4 may provide additional balance between current output through the electrode elements 402, since all electrode elements 402 are positioned to radiate substantially symmetrically outward from the center portion 411 of the array. In addition, the rounded edges 414 trace the rounded outer perimeter 406 of the array. This eliminates corners in the overall shape of the array, which may prevent high concentrations of current due to the edge effect.

In each electrode element layout described herein (e.g., in FIGS. 4 and 6-10), any number of electrode elements 402 in the array may have substantially similar shapes. For example, in FIG. 4, all electrode elements 402A-402H have substantially similar shapes as described above. In other embodiments (FIGS. 4 and 6-10), one or more electrode elements in the array may have substantially different shapes from one another. As depicted in FIG. 4, each electrode element 402A-402H in the array may have approximately the same surface area, further balancing the current output from individual electrode elements.

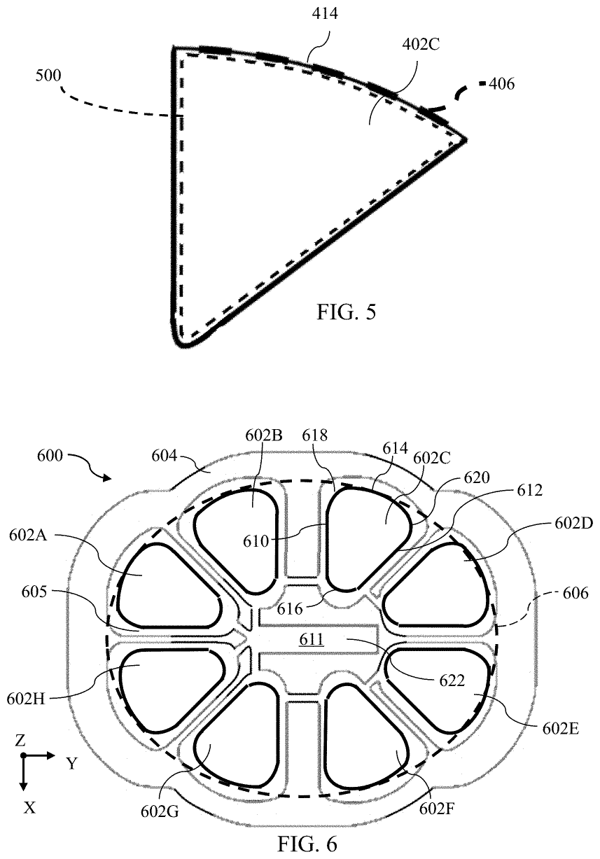

FIG. 5 illustrates in greater detail the electrode element 402C of FIG. 4. FIG. 5 depicts a perimeter 500 of the electrode element 402C (in small dashed lines) and the portion of the outer perimeter 406 that is touching the electrode element 402C (in large dashed lines). As depicted, at least 10% of the length of the perimeter 500 of the electrode element 402C is touching the outer perimeter 406 (e.g., along the curved edge 414). In an example transducer, each electrode element 402 touching the outer perimeter 406 may have at least 10% of the length of their perimeter 500 touching the outer perimeter 406. For each of the embodiments described herein, each electrode element touching the outer perimeter of the array may have at least 30%, at least 20%, at least 15%, at least 10%, or at least 5% of the length of their perimeter touching the outer perimeter of the array, such as, for example, from 5% to 30%, or from 10% to 15%, or from 10% to 20%, or from 10% to 30% of the length of their perimeter touching the outer perimeter of the array. A substantial portion of each peripheral electrode element is thus following the edge of the outer perimeter 406, providing a more balanced distribution of current along the edge of the transducer compared to transducers with electrodes (e.g., disk-shaped electrodes) that touch the outer perimeter only at discrete points.

Turning back to FIG. 4, it may be desirable for an electrode element of the array to touch at least a certain percentage of the total length of the outer perimeter 406. For example, at least one electrode element 402C in the array has a curved edge 414 that touches a curved section of the outer perimeter 406 along at least 5% of the length of the outer perimeter 406. For each of the embodiments described herein, at least one electrode element in the array has a curved edge that touches a curved section of the outer perimeter along at least 30%, at least 20%, at least 15%, at least 10%, or at least 5% of the length of the outer perimeter, such as, for example, from 5% to 10%, or from 5% to 15%, or from 5% to 20%, or from 10% to 30% of the length of the outer perimeter. In an example, at least 50% of a total number of the electrode elements in an array may have a curved edge that touches a curved section of the outer perimeter along at least 5% of the length of the outer perimeter. For each of the embodiments described herein, at least 50%, at least 60%, at least 70%, at least 80%, or at least 90% of a total number of the electrode elements in an array may have a curved edge that touches a curved section of the outer perimeter along at least 5% of the length of the outer perimeter. For example, from 50% to 60%, or from 50% to 70%, or from 50% to 80%, or from 50% to 90% of a total number of the electrode elements in an array may have a curved edge that touches a curved section of the outer perimeter along at least 5% of the length of the outer perimeter. Further, at least 50%, at least 60%, at least 70%, at least 80%, or at least 90% of a total number of the electrode elements in an array may have a curved edge that touches a curved section of the outer perimeter along at least 30%, at least 20%, at least 15%, at least 10%, or at least 5% of the length of the outer perimeter, and within the same associated ranges. In an example, at least six electrode elements in an array may have a curved edge that touches a curved section of the outer perimeter along at least 5% of the outer perimeter. In FIG. 4, every electrode element in the array has a curved edge (e.g., 414) that touches a curved section of the outer perimeter 406 along at least 5% of the length of the outer perimeter 406. This helps spread the electrode elements 402 out along the rounded convex perimeter so that the overall shape of the array of electrode elements 402 is rounded, without corners.

In an example, at least 30% of the total length of the outer perimeter 406 touches one or more electrode elements 402 in the array. Even further, at least 50% of the length of the outer perimeter 406 touches one or more electrode elements 402 in the array. As depicted in FIG. 4, due to the shape of the individual electrode elements 402, at least 60%, more particularly at least 80%, and more particularly at least 90%, of the length of the outer perimeter 406 may touch electrode elements 402 of the array. Increasing or maximizing the amount of the outer perimeter 406 that is touching electrode elements in this manner may further balance current output through the array by conforming a large portion of the electrode elements' edges to a rounded shape. For each of the embodiments described herein, at least 20%, at least 30%, at least 40%, at least 50%, at least 60%, at least 70%, at least 80%, or at least 90% of the total length of the outer perimeter touches one or more electrode elements in the array, such as, for example, from 30% to 60%, or from 30% to 70%, or from 30% to 80%, or from 30% to 90% or from 50% to 60%, or from 50% to 70%, or from 50% to 80%, or from 50% to 90% of the total length of the outer perimeter touches one or more electrode elements in the array.

FIG. 6 depicts a transducer 600 with an example layout of electrode elements 602, which may be disposed on a substrate 604. The layout of electrode elements 602 is similar to the layout of FIG. 4, but with differently shaped and unevenly spaced electrode elements 602. In addition, a layered structure of the transducer 600 is depicted in FIG. 6. As shown, the transducer 600 may include a printed circuit board (PCB) level 605 between the electrode elements 602 and the substrate 604. The PCB level 605 may include conductive pathways that electrically couple the electrode elements 602 together. The PCB level 605 may include an electrical connector portion 622 that provides a point for connecting leads to the transducer 600. As illustrated, the electrical connector portion 622 may be disposed at a center portion 611 of the transducer 600, surrounded by the electrode elements 602 of the array. Other embodiments of the transducer may feature an electrical connector portion that is located elsewhere on the transducer.

In FIG. 6, the transducer's array of electrode elements comprises eight electrode elements 602A-602H, all of which are peripheral electrode elements. An outer perimeter 606 of the array is shown in dashed lines. The outer perimeter 606 is a rounded convex perimeter substantially tracing the electrode elements 602 of the array. As depicted, the outer perimeter 606 circumscribes the array of electrode elements 602. In the embodiment of FIG. 6, the outer perimeter 606 touches an edge of every electrode element 602A-602H in the array. At least a portion of a length of the perimeter of each peripheral electrode element 602A-602H is touching the outer perimeter 606.

The electrode elements 602 depicted in FIG. 6 each have a substantially wedge shape with rounded corners. The electrode elements 602 each have a radially internal facing rounded corner (e.g., 616) and a radially external facing rounded edge (e.g., 614) between the two remaining corners. One or more electrode elements 602 may comprise: a first edge 610 extending in a radially outward direction relative to the center portion 611 of the array; a second edge 612 extending in a radially outward direction relative to the center portion 611 of the array; and a rounded edge 614 connecting the first edge 610 to the second edge 612 at an end of the electrode element located radially away from the center portion 611 of the array. As illustrated, a rounded corner 616 may connect the first edge 610 to the second edge 612 at an opposite end of the electrode element located radially toward the center portion 611. As depicted, a rounded corner 618 may connect the first edge 610 to the rounded edge 614, and another rounded corner 620 may connect the second edge 612 to the rounded edge 614. FIG. 6 depicts all the electrode elements 602 having substantially similar shapes as described above. However, in other embodiments, one or more electrode elements in the array may have substantially different shapes from one another. Each electrode element 602 in the array may have approximately the same surface area.

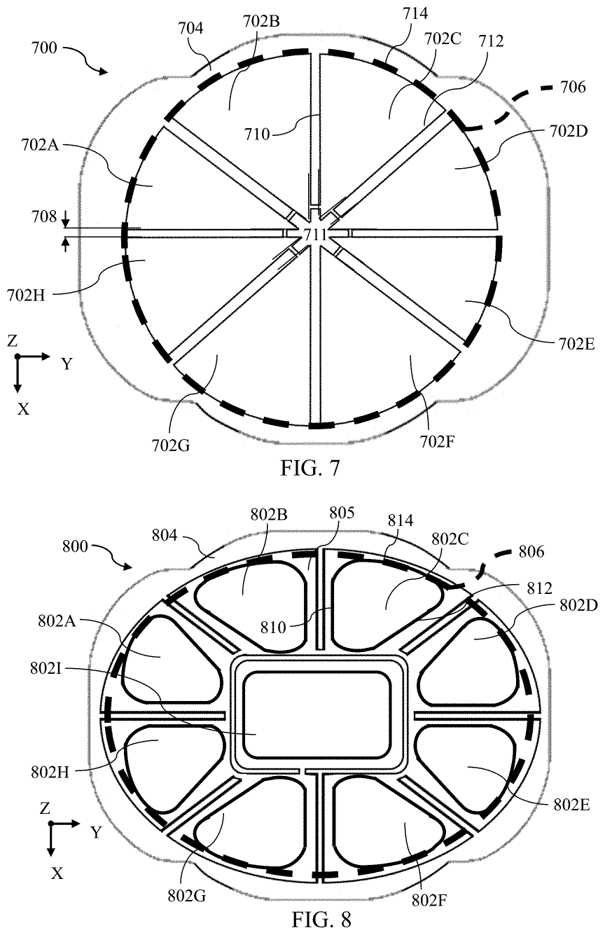

FIG. 7 depicts a transducer 700 with an example layout of electrode elements 702, which are coupled to each other and may be disposed on a substrate 704. The layout of electrode elements 702 is similar to the layout of FIG. 4, but with differently shaped electrode elements 702 and a differently shaped outer perimeter 706. In FIG. 7, the transducer's array of electrode elements comprises eight electrode elements 702A-702H, all peripheral electrode elements.

FIG. 7 depicts an outer perimeter 706, which is a rounded convex perimeter substantially tracing the electrode elements 702 of the array. The outer perimeter 706 is defined by a form-fit convex shape surrounding the plurality of electrode elements 702, and therefore circumscribes the array of electrode elements 702. The outer perimeter 706 may be circular. As depicted, the outer perimeter 706 may be shaped such that every point along the outer perimeter 706 is equidistant from a point (e.g., a centroid of the array) inside the outer perimeter 706.

The outer perimeter 706 touches an edge of every electrode element 702A-702H in the array. As depicted, at least a portion of a length of a perimeter of each electrode element (702A-702H) is touching the outer perimeter 706. In particular, the outer perimeter 706 traces one or more curved edges (e.g., curved edge 714) of the electrode elements 702 touching the outer perimeter 706.

In an example, the electrode elements 702A-702H in the array may be spaced substantially equidistant from each other about the array. In FIG. 7, for example, each pair of adjacent peripheral electrode elements (e.g., 702A and 702H) touching the outer perimeter 706 may have approximately a same distance (e.g., 708) therebetween, as described above with respect to the distance 408 in FIG. 4.

FIG. 7 depicts each of the electrode elements 702 having a wedge shape with a radially external facing rounded edge (e.g., 714). As illustrated with reference to the electrode element 702C, one or more electrode elements 702 may comprise: a first edge 710 extending in a radially outward direction relative to the center portion 711 of the array; a second edge 712 extending in a radially outward direction relative to the center portion 711 of the array; and a rounded edge 714 connecting the first edge 710 to the second edge 712 at an end of the electrode element located radially away from the center portion 711 of the array. Any number of electrode elements 702 in the array may have substantially similar shapes. For example, in FIG. 7, all electrode elements 702 have substantially similar shapes as described above. However, in other embodiments, one or more electrode elements in the array may have substantially different shapes from one another. Each electrode element 702 in the array may have approximately the same surface area.

At least one electrode element 702 in the array of FIG. 7 has a curved edge 714 that touches a curved section of the outer perimeter 706 along at least 5% of the length of the outer perimeter 706. As depicted in FIG. 7, every electrode element in the array has a curved edge (e.g., 714) that touches a curved section of the outer perimeter 706 along at least 5% of the length of the outer perimeter 406. At least 30%, more particularly at least 50%, of the total length of the outer perimeter 706 in FIG. 7 touches one or more electrode elements 702 in the array.

FIG. 8 depicts a transducer 800 with an example layout of electrode elements 802, which are coupled to each other and may be disposed on a substrate 804. The transducer 800 depicted in FIG. 8 has a PCB layer 805, similar to the PCB layer described with reference to FIG. 6. The layout of electrode elements 802 is similar to the layout of FIG. 6, but with differently shaped and differently arranged electrode elements 802. In FIG. 8, the transducer's array of electrode elements comprises nine electrode elements 802A-8021, including eight peripheral electrode elements 802A-802H and one non-peripheral electrode 8021. As illustrated, at least one electrode element (e.g., 8021) in the array may be surrounded by one or more peripheral electrode elements of the array and does not touch the outer perimeter 806.

In FIG. 8, the outer perimeter 806 is a rounded convex perimeter substantially tracing the electrode elements 802 of the array. The outer perimeter 806 touches an edge of every peripheral electrode element 802A-802H. As depicted, at least a portion of a perimeter of each peripheral electrode element (802A-802H) is touching the outer perimeter 806.

The peripheral electrode elements 802 depicted in FIG. 8 each have a substantially truncated wedge shape with rounded corners. One or more electrode elements 802 may comprise: a first edge 810 extending in a radially outward direction relative to the center portion of the array; a second edge 812 extending in a radially outward direction relative to the center portion of the array; and a rounded edge 814 connecting the first edge 810 to the second edge 812 at an end of the electrode element located radially away from the center portion of the array. In some embodiments, all the peripheral electrode element(s) may have substantially similar shapes. In other embodiments, one or more of the peripheral electrode elements may have substantially different shapes from one another. The non-peripheral electrode element 8021 depicted in FIG. 8 has a substantially rectangular shape with rounded corners and is disposed in the center portion of the array. Other numbers (e.g., 2, 3, 4, 5, 6, 7, 8, 9, 10, or more) of non-peripheral electrode elements may be included in other embodiments. Non-peripheral electrode element(s) may take any desired shape including, but not limited to, a square, rectangular, hexagonal, or polygonal shape, a substantially square, rectangular, hexagonal, or polygonal shape with one or more rounded corners, an irregular shape, or a circular, oval, ovaloid, ovoid, or elliptical shape. In some embodiments, there may be more than one non-peripheral electrode element. In some embodiments, all the non-peripheral electrode element(s) may have substantially similar shapes. In other embodiments, one or more of the non-peripheral electrode elements may have substantially different shapes from one another.

FIG. 9 depicts a transducer 900 with an example layout of electrode elements 902, which are coupled to each other and may be disposed on a substrate 904. The transducer 900 depicted in FIG. 9 has a PCB layer 905, which may include an electrical connector portion 922 to provide a point for connecting leads to the transducer 900. The layout of electrode elements 902 is similar to the layout of FIG. 6, but with differently shaped/arranged electrode elements 902 and a differently shaped outer perimeter 906. In FIG. 9, the transducer's array of electrode elements comprises eight electrode elements 902A-902H, all peripheral electrode elements.

FIG. 9 depicts an outer perimeter 906, which is a rounded convex perimeter substantially tracing the electrode elements 902 of the array. The outer perimeter 906 circumscribes the array of electrode elements 902. As depicted, the outer perimeter 906 may be rectangular with rounded corners. In an example, the outer perimeter 906 may be shaped such that, at each rounded corner of the outer perimeter 906, every point along the rounded corner portion is equidistant from a point inside the outer perimeter 906. The outer perimeter 906 touches an edge of every electrode element 902A-902H in the array. As depicted, at least a portion of a perimeter of each peripheral electrode element (902A-902H) is touching the outer perimeter 906. FIG. 9 depicts each of the electrode elements 902 having a substantially rectangular shape with rounded corners.

FIG. 10 depicts a transducer 1000 with an example layout of electrode elements 1002, which are coupled to each other and may be disposed on a substrate 1004. The transducer 1000 depicted in FIG. 10 has a PCB layer 1005, which may include an electrical connector portion 1022 to provide a point for connecting leads to the transducer 1000. The layout of electrode elements 1002 is similar to the layout of FIG. 6, but with the electrode elements 1002 located in different positions. In FIG. 10, the transducer's array of electrode elements comprises eight electrode elements 1002A-1002H, all of which are peripheral electrode elements. In some embodiments, all the electrode element(s) may have substantially similar shapes. In other embodiments, one or more of the electrode elements may have substantially different shapes from one another.

FIG. 10 depicts an outer perimeter 1006, which is a rounded convex perimeter circumscribing the array of electrode elements 1002. As depicted, the outer perimeter 1006 touches or extends adjacent an edge of every electrode element 1002 in the array. For example, the outer perimeter 1006 touches the electrode elements 1002A, 1002D, 1002E, and 1002H. The outer perimeter 1006 extends adjacent to an edge of each of the electrode elements 1002B, 1002C, 1002F, and 1002G. As depicted in FIG. 10, every electrode element 1002A-1002H in the array has an edge located less than a certain distance away from the outer perimeter 1006. For example, a distance 1024 from the electrode element 1002B to the outer perimeter 1006 may be less than 20% of the length of the perimeter of the electrode element 1002B. The electrode elements 1002C, 1002F, and 1002G may similarly be a distance less than this amount from the outer perimeter 1006. The other electrode elements 1002A, 1002D, 1002E, and 1002H touch the outer perimeter 1006 and so their edge is no distance away from the outer perimeter 1006. For each of the embodiments of arrays disclosed herein, an embodiment exists for which every electrode element in the array has an edge located a distance of less than 30%, or less than 20%, or less than 10%, or less than 5%, or less than 2%, or less than 1% of the perimeter of the electrode element away from the outer perimeter circumscribing the array, such as, for example, a distance of from 1% to 30%, or from 1% to 20%, or from 1% to 10%, or from 1% to 5% or from 5% to 30%, or from 5% to 20%, or from 5% to 10% of the perimeter of the electrode element away from the outer perimeter circumscribing the array.

In an example, the electrode elements 1002A-1002H in the array may be spaced substantially equidistant from each other about the array. In FIG. 10, for example, each pair of adjacent electrode elements (e.g., 1002A and 1002H) in the array may have approximately a same distance (e.g., 1008) therebetween, as described above with respect to the distance 408 in FIG. 4.

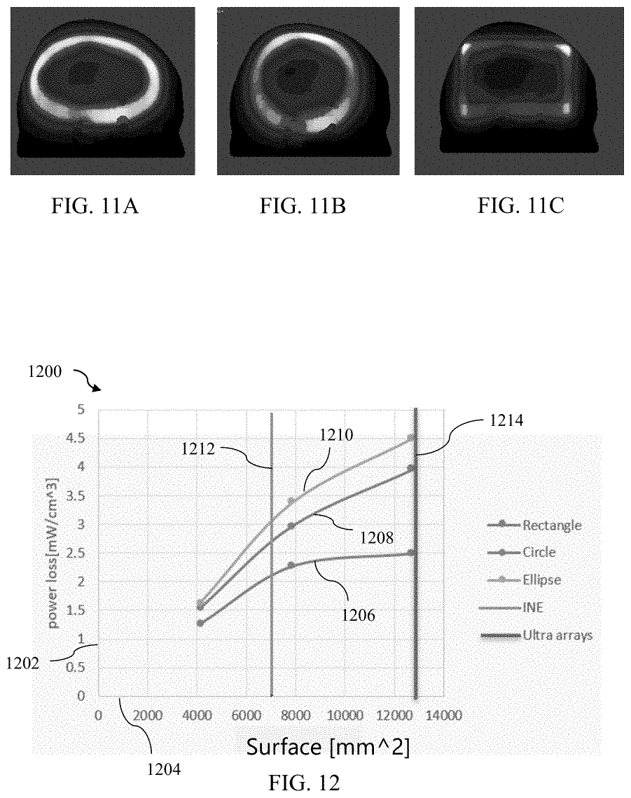

FIGS. 11A, 11B, and 11C show the Specific Absorption Rate (SAR) under the array on the scalp for arrays of electrode elements having different outer perimeter shapes. The SAR measures the energy absorbed by biological tissues and provides an estimate of the temperature rise induced at the tissue. At a given location, the SAR is computed as the ratio between the dissipated power and the mass densities as provided in Equation (1):

$$SAR ( r ) = \frac{σ}{2 ρ} {E}^{2} ∝ \frac{d T}{dt} \qquad Equation ( 1 )$$

where σ is the electrical conductivity of the tissue, E denotes the magnitude of the induced electric field, ρ is the mass density (kg/m³) and Tis the temperature (degrees Kelvin).

FIG. 11A shows the SAR under an array having an oval or elliptical outer perimeter, FIG. 11B shows the SAR under an array having a circular outer perimeter, and FIG. 11C shows the SAR under an array having a rectangular outer perimeter. The images for all three shapes show the same maximum SAR value, because the SAR is representative of temperature, and the same maximum temperature is used to simulate the temperature threshold that exists in actual use. As depicted in FIGS. 11A and 11B, the SAR under both the elliptical and circular arrays is relatively consistent along the entire outer edge of the array. Similar results may be seen from arrays having substantially ovoid or ovaloid outer perimeters. A maximum SAR (corresponding to maximum temperature) all over the area for the elliptical and circular arrays results in a higher current delivered by the array. However, for the rectangular array, the ‘hot spots’ occur only at the corners and the black (cooler) area in the center is much larger indicating the majority of the charge is concentrated in the corners resulting in a less active treatment area. The rising temperatures in the corners limit the current delivered by the array.

Moving from substantially rectangular arrays (e.g., the rectangular 3×3 array of FIG. 3C, or the rectangular array depicted in FIG. 11C) to a substantially circular, oval, ovoid, ovaloid, or elliptical array (e.g., FIGS. 11A and 11B) may reduce or minimize the edge effect, thereby decreasing or eliminating hot spots. By removing corners in the overall shape of the electrode element arrays, the disclosed transducers provide a more uniform electric field strength around the edge, allowing stronger TTFields to be induced without overheating the subject's body.

FIG. 12 depicts a plot 1200 of average power loss 1202 (mW/cm³) with respect to array surface area 1204 (mm²) for electrode arrays having different outer perimeter shapes (rectangle, circle, and ellipse). The TTFields power loss density represents the energy per unit of time deposited by the TTFields within the body. For each electrode array shape, the relationship depicted in the plot 1200 was determined by simulating the average power loss 1202 through the brain from arrays having three different surface areas 1204 (e.g., 4,160 mm², 7,865 mm², and 12,740 mm²). The simulated average power loss is proportional to the squared magnitude of the electric field strength of the TTFields output by the array as provided in Equation (2):

power loss=0.5σE² Equation (2)

where σ is the electrical conductivity of the tissue, and E denotes the magnitude of the induced electric field The results from the simulations are depicted in the plot 1200. Trend line 1206 represents the relationship between average power loss 1202 and array surface area 1204 for rectangular shaped arrays. Trend line 1208 represents the relationship between average power loss 1202 and array surface area 1204 for circular shaped arrays. Trend line 1210 represents the relationship between average power loss 1202 and array surface area 1204 for elliptical shaped arrays. As depicted, the elliptical shaped arrays (1210) have the highest power loss 1202 for each surface area 1204, the rectangular shaped arrays (1206) have the lowest power loss 1202 for each surface area 1204, and the circular shaped arrays (1208) have a power loss 1202 between that of the elliptical and rectangular arrays. This means that the elliptical arrays 1210 are able to induce stronger TTFields than circular arrays 1208, and the circular arrays 1208 are able to induce stronger TTFields than rectangular arrays 1206, at the same temperatures. The rectangular arrays 1206 provide the lowest performance due to current/heat concentrations (hot spots) that occur at their four corners due to the edge effect.

Table 1 below shows the differential power loss (in percentages) between the different shaped arrays for each surface area.

TABLE 1
Differential power loss between different shaped arrays

| | Differential | Differential | Differential |
|---|---|---|---|
| | power loss from | power loss from | power loss from |
| Surface | rectangle to | rectangle to | circle to |
| area (mm²) | circle shape | ellipse shape | ellipse shape |
| 4,160 | 21.2% | 28.7% | 6.2% |
| 7,856 | 30.7% | 48.8% | 13.9% |
| 12,740 | 58.6% | 80.6% | 13.9% |

As shown in Table 1, when the transducer is small (e.g., lower array surface area), the differences between the rectangle, circle, and ellipse array shapes are much less pronounced compared to when the transducer is large (e.g., higher array surface area). The greatest difference in power loss is between the rectangular and elliptical shaped arrays, at any surface area but particularly at the largest surface area (12,740 mm²).

The results of the simulations show that increasing the surface area of an array having the same array shape may be a less efficient way to increase TTField strength than simply changing the array shape for the same transducer surface area. The plot 1200 shows a vertical line 1212 representing the size of the surface area of a first standard array, “INE” (“Insulated Nine Electrodes”) and another vertical line 1214 representing the size of the surface area of a second standard array (“Ultra array”). Increasing a rectangular array 1206 from the INE surface area size (1212) to the Ultra array surface area size (1214) may provide up to a 20% gain in power loss as shown on the plot 1200. However, simply changing from a rectangular array 1206 to an elliptical array 1210 at the same INE size (1212) may provide up to a 50% gain in power loss. Similarly, changing the array shape from a rectangle to an ellipse in an area of 7,865 mm² (˜INE size) increases the average power loss in the brain by 36% more than increasing the rectangular area from 7,865 mm² to 12,740 mm² (˜Ultra array size).

The invention includes other items, such as the following.

Item 1: A transducer apparatus for delivering tumor treating fields to a subject's body, the transducer apparatus comprising: an array of electrode elements electrically coupled to each other, the array comprising all electrode elements present on the transducer apparatus, the array configured to be positioned over the subject's body with a face of the array facing the subject's body; wherein, when viewed from a direction perpendicular to the face of the array, an outer perimeter of the array substantially tracing the electrode elements of the array has a rounded convex shape; a number of the electrode elements of the array are peripheral electrode elements defining the outer perimeter of the array, the peripheral electrode elements substantially surrounding any other electrode elements of the array; wherein for each peripheral electrode element, at least a portion of a length of a perimeter of the peripheral electrode element is touching the outer perimeter of the array.

Item 2: The transducer apparatus of Item 1, wherein the outer perimeter does not have any corners. Item 3: The transducer apparatus of Item 1, wherein the outer perimeter is substantially circular, oval, ovaloid, ovoid, or elliptical. Item 4: The transducer apparatus of Item 1, wherein a portion of the outer perimeter is shaped such that every point along the portion of the outer perimeter is equidistant from a point inside the outer perimeter. Item 5: The transducer apparatus of Item 1, wherein at least one of the electrode elements in the array has a triangular shape, a substantially triangular shape with rounded corners, a truncated triangular shape, a substantially truncated triangular shape with rounded corners, a wedge shape, a substantially wedge shape with rounded corners, a truncated wedge shape, or a substantially truncated wedge shape with rounded corners. Item 6: The transducer apparatus of Item 1, wherein at least one of the electrode elements in the array comprises: a first edge extending in a radially outward direction relative to the center portion of the array; a second edge extending in a radially outward direction relative to the center portion of the array; and a rounded edge connecting the first edge to the second edge at an end of the electrode element located radially away from the center portion of the array. Item 7: The transducer apparatus of Item 1, wherein each electrode element in the array is a peripheral electrode element touching the outer perimeter. Item 8: The transducer apparatus of Item 1, wherein at least one electrode element in the array is surrounded by one or more peripheral electrode elements of the array and does not touch the outer perimeter. Item 9: The transducer apparatus of Item 1, wherein for each of the peripheral electrode elements, at least 10% of the length of the perimeter of the peripheral electrode element is touching the outer perimeter. Item 10: The transducer apparatus of Item 1, wherein the array of electrode elements are capacitively coupled. Item 11: The transducer apparatus of Item 1, wherein the array of electrode elements are not capacitively coupled. Item 12: The transducer apparatus of Item 1, wherein the electrode elements comprise a ceramic dielectric layer. Item 13: The transducer apparatus of Item 1, wherein the electrode elements comprise polymer films.

Item 14: A transducer apparatus for delivering tumor treating fields to a subject's body, the transducer apparatus comprising: a plurality of electrode elements electrically coupled to each other and forming an array in a plane of the transducer apparatus; wherein, when viewed from a direction perpendicular to the plane: an outer perimeter of the array is defined by a form-fit convex shape surrounding the plurality of electrode elements; and at least 30% of the length of the outer perimeter touches one or more electrode elements of the plurality of electrode elements.

Item 15: The transducer apparatus of Item 14, wherein, when viewed from the direction perpendicular to the plane, at least 50% of the length of the outer perimeter touches one or more electrode elements of the plurality of electrode elements. Item 16: The transducer apparatus of Item 14, wherein the outer perimeter traces one or more curved edges of the one or more electrode elements touching the outer perimeter. Item 17: The transducer apparatus of Item 14, wherein for each of the one or more electrode elements touching the outer perimeter, at least 10% of the length of a perimeter of the electrode element is touching the outer perimeter. Item 18: The transducer apparatus of Item 14, wherein the outer perimeter has a substantially circular, oval, ovaloid, ovoid, or elliptical shape.

Item 19: A transducer apparatus for delivering tumor treating fields to a subject's body, the transducer apparatus comprising: an array of electrode elements electrically coupled to each other, the array comprising all electrode elements present on the transducer apparatus, the array configured to be positioned over the subject's body with a face of the array facing the subject's body; wherein, when viewed from a direction perpendicular to the face of the array: an outer perimeter circumscribing the array of electrode elements has a substantially circular, oval, ovaloid, ovoid, or elliptical shape; and at least one electrode element in the array of electrode elements has a curved edge that touches a curved section of the outer perimeter along at least 5% of the length of the outer perimeter.

Item 20: The transducer apparatus of Item 19, wherein at least 50% of a total number of electrode elements in the array of electrode elements have a curved edge that touches a curved section of the outer perimeter along at least 5% of the length of the outer perimeter. Item 21: The transducer apparatus of Item 19, wherein at least six electrode elements in the array of electrode elements have a curved edge that touches a curved section of the outer perimeter along at least 5% of the length of the outer perimeter. Item 22: The transducer apparatus of Item 19, wherein every electrode element in the array of electrode elements has a curved edge that touches a curved section of the outer perimeter along at least 5% of the length of the outer perimeter.

Item 23: A transducer apparatus for delivering tumor treating fields to a subject's body, the transducer apparatus comprising: an array of electrode elements electrically coupled together, the array configured to be positioned over the subject's body with a face of the array facing the subject's body; wherein, when viewed from a direction perpendicular to the face of the array, an outer perimeter circumscribing the array of electrode elements to be positioned over the subject's body touches or extends adjacent an edge of every electrode element in the array.

Item 24: The transducer apparatus of Item 23, wherein, when viewed from the direction perpendicular to the face of the array, every electrode element in the array has an edge located a distance less than 20% of the perimeter of the electrode element away from the outer perimeter circumscribing the array. Item 25: The transducer apparatus of claim 23, wherein the electrode elements of the array are spaced substantially equidistant from each other about the array.

Item 26: A transducer apparatus for delivering tumor treating fields to a subject's body, the transducer apparatus comprising: an array of electrode elements electrically coupled together, the array configured to be positioned over the subject's body with a face of the array facing the subject's body; wherein, when viewed from a direction perpendicular to the face of the array, an outer perimeter circumscribing the array of electrode elements to be positioned over the subject's body has a rounded convex shape, and wherein each electrode element in the array either has an edge that touches the perimeter or has an edge located a distance less than 20% of the perimeter of the electrode element away from the outer perimeter circumscribing the array.

Item 27: The transducer apparatus of Item 26, wherein the electrode elements of the array are spaced substantially equidistant from each other about the array.

Item 28: A transducer apparatus according to any of Items 1-27, wherein the array of electrode elements comprises at least six electrode elements. Item 29: A transducer apparatus according to any of Items 1-27, wherein each electrode element has approximately the same surface area. Item 30: A transducer apparatus according to any of Items 1-27, wherein the outer perimeter is substantially rectangular with rounded corners.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
an array of electrode elements electrically coupled to each other, the array comprising all electrode elements present on the transducer apparatus, the array configured to be positioned over the subject's body with a face of the array facing the subject's body;
wherein, when viewed from a direction perpendicular to the face of the array, an outer perimeter of the array substantially tracing the electrode elements of the array has a rounded convex shape;
a number of the electrode elements of the array are peripheral electrode elements defining the outer perimeter of the array, the peripheral electrode elements substantially surrounding any other electrode elements of the array;
wherein for each peripheral electrode element, at least a portion of a length of a perimeter of the peripheral electrode element is touching the outer perimeter of the array.

2. The transducer apparatus of claim 1, wherein the outer perimeter does not have any corners.

3. The transducer apparatus of claim 1, wherein the outer perimeter is substantially circular, oval, ovaloid, ovoid, or elliptical.

4. The transducer apparatus of claim 1, wherein a portion of the outer perimeter is shaped such that every point along the portion of the outer perimeter is equidistant from a point inside the outer perimeter.

5. The transducer apparatus of claim 1, wherein at least one of the electrode elements in the array has a triangular shape, a substantially triangular shape with rounded corners, a truncated triangular shape, a substantially truncated triangular shape with rounded corners, a wedge shape, a substantially wedge shape with rounded corners, a truncated wedge shape, or a substantially truncated wedge shape with rounded corners.

6. The transducer apparatus of claim 1, wherein at least one of the electrode elements in the array comprises:
a first edge extending in a radially outward direction relative to the center portion of the array;
a second edge extending in a radially outward direction relative to the center portion of the array; and
a rounded edge connecting the first edge to the second edge at an end of the electrode element located radially away from the center portion of the array.

7. The transducer apparatus of claim 1, wherein each electrode element in the array is a peripheral electrode element touching the outer perimeter.

8. The transducer apparatus of claim 1, wherein at least one electrode element in the array is surrounded by one or more peripheral electrode elements of the array and does not touch the outer perimeter.

9. The transducer apparatus of claim 1, wherein for each of the peripheral electrode elements, at least 10% of the length of the perimeter of the peripheral electrode element is touching the outer perimeter.

10. The transducer apparatus of claim 1, wherein the array of electrode elements are capacitively coupled.

11. The transducer apparatus of claim 1, wherein the array of electrode elements are not capacitively coupled.

12. The transducer apparatus of claim 1, wherein the electrode elements comprise a ceramic dielectric layer.

13. The transducer apparatus of claim 1, wherein the electrode elements comprise polymer films.

14. A transducer apparatus for delivering tumor treating fields to a subject's body, the transducer apparatus comprising:
a plurality of electrode elements electrically coupled to each other and forming an array in a plane of the transducer apparatus;
wherein, when viewed from a direction perpendicular to the plane:
an outer perimeter of the array is defined by a form-fit convex shape surrounding the plurality of electrode elements; and
at least 30% of the length of the outer perimeter touches one or more electrode elements of the plurality of electrode elements.

15. The transducer apparatus of claim 14, wherein the outer perimeter traces one or more curved edges of the one or more electrode elements touching the outer perimeter.

16. The transducer apparatus of claim 14, wherein for each of the one or more electrode elements touching the outer perimeter,
at least 10% of the length of a perimeter of the electrode element is touching the outer perimeter.

17. The transducer apparatus of claim 14, wherein the outer perimeter has a substantially circular, oval, ovaloid, ovoid, or elliptical shape.

18. A transducer apparatus for delivering tumor treating fields to a subject's body, the transducer apparatus comprising:
an array of electrode elements electrically coupled to each other, the array comprising all electrode elements present on the transducer apparatus, the array configured to be positioned over the subject's body with a face of the array facing the subject's body;
wherein, when viewed from a direction perpendicular to the face of the array:
an outer perimeter circumscribing the array of electrode elements has a substantially circular, oval, ovaloid, ovoid, or elliptical shape; and
at least one electrode element in the array of electrode elements has a curved edge that touches a curved section of the outer perimeter along at least 5% of the length of the outer perimeter.

19. The transducer apparatus of claim 18, wherein at least 50% of a total number of electrode elements in the array of electrode elements have a curved edge that touches a curved section of the outer perimeter along at least 5% of the length of the outer perimeter.

20. The transducer apparatus of claim 18, wherein every electrode element in the array of electrode elements has a curved edge that touches a curved section of the outer perimeter along at least 5% of the length of the outer perimeter.
